(19) United States
(12) Patent Application Publication
(10) Pub. No.: US 2020/0327963 A1
(43) Pub. Date: Oct. 15, 2020

(54) Latent Space Exploration Using Linear-Spherical Interpolation Region Method

(71) Applicant: ACCENTURE GLOBAL SOLUTIONS LIMITED, Dublin (IE)

(72) Inventors: Qurrat Ul Ain, Dublin (IE); Nicholas McCarthy, Dublin (IE); Jeremiah Hayes, Dublin (IE); Philip O'Kelly, Skerries (IE); Patrick Moreau, Dublin (IE)

(73) Assignee: ACCENTURE GLOBAL SOLUTIONS LIMITED, Dublin (IE)

(21) Appl. No.: 16/445,811

(22) Filed: Jun. 19, 2019

Related U.S. Application Data
(60) Provisional application No. 62/832,489, filed on Apr. 11, 2019.

(51) Int. Cl. G16C 20/30; G16H 20/10; G16B 15/30

(52) U.S. Cl. CPC G16C 20/30; G16B 15/30; G16H 20/10

(57) ABSTRACT

The disclosure enables latent space exploration of a dataset based on drug molecular-structure data and drug biological-treatment data for a set of drug compounds in order to determine optimal drug compounds for treating diseases. Regional interpolation, including a linear interpolation (LERP) operation and a non-linear interpolation operation such as a spherical linear interpolation (SLERP), along with quantitative structure-activity relationship (QSAR) models may be utilized to navigate through a latent space generated from a variational auto-encoder (VAE), in accordance with certain embodiments.

### CROSS-REFERENCE TO RELATED APPLICATION(S)

This application claims benefit to U.S. Provisional Patent Application No. 62/832,489, filed on Apr. 11, 2019, the entirety of which is incorporated by reference herein.

### FIELD OF THE INVENTION

The present disclosure relates in general to the fields of bioinformatics and latent space exploration, and in particular methods and systems for identifying drug compounds for experimental usage in the treatment of diseases using latent space generated by variational auto-encoders based on the combination of drug molecular-structure data and drug biological-treatment data.

### BACKGROUND

Basic techniques and equipment for ranking drug compounds, scoring gene expressions and enrichment pathways, and selecting predictive biomarkers are known in the art. Both drug data and biological data have features that can be described as discrete values using variational auto-encoders to generate latent spaces for modeling the metrics of latent variables that may be explored using interpolation methods and quantitative structure-activity relationship models. While various technologies have used either drug data or biological data independently to generate latent space, a multi-modal latent space based on the combination of drug molecular-structure data and biological-treatment data is desired to more efficiently identify optimal and/or new drug compounds for the treatment of diseases.

### BRIEF SUMMARY

The present disclosure may be embodied in various forms, including without limitation a system, a method or a computer-readable medium for latent space exploration using regional interpolation and quantitative structure-activity relationship (QSAR) models to navigate through a latent space generated from an encoder, such as a variational auto-encoder (VAE). The latent space may graphically represent embedding vectors, where each embedding vector may comprise a metric representation of a plurality of drug compounds and attributes associated with the drug compounds. In an embodiment, each embedding vector may correspond to a probability measurement or metric associated with the drug compounds and their latent attributes. Latent attributes may comprise “hidden” data that would not have otherwise been observed and considered, without the use of the present disclosure. These latent attributes may be used to identify candidate compounds or molecules that may be used as new drugs to treat various diseases, in accordance with certain embodiments. In order to determine a list of optimal candidates, a regional interpolation method and a QSAR model may be utilized to determine an optimal path between two clusters of nodes in the latent space. Each node in the latent space may correspond to an embedding vector representing the metrics for a drug compound and the various attributes of that drug compound. The optimal path may include nodes representing the top-ranked candidate points associated with the treatment of certain diseases.

In an embodiment, the regional interpolation method utilized to determine the optimal path in the latent space may comprise a linear interpolation and a non-linear interpolation, such as spherical interpolation, circular interpolation, or elliptical interpolation. While the clusters of nodes may represent a region of interest in the latent space corresponding to the patent attributes for drug compounds known to be associated with the biological-treatment data for certain diseases, the optimal path of nodes may correspond to latent attributes that may have not yet been considered in the selection of drug compounds for the treatment of such diseases. The optimal path between two clusters may represent candidates for drug formulations, which may comprise either pre-existing or new compounds, that may be effective against the diseases associated with the two clusters. In some embodiments, the targeted clusters may be identified by a query performed on the entire set of vectors embedded in the latent space. The query may include a drug molecular-structure query, a drug treatment query, and/or a drug effect query. A cluster of nodes in the latent space identified by the query may be annotated or marked with a disease label that corresponds to a certain disease. In an embodiment, the labelled nodes may represent drug compounds known to be effective in the treatment of the disease.

In some embodiments, a latent space may be generated via variational auto-encoders based on pre-existing drug data and human biological data for a plurality of drug compounds. This “input” data may include structural information for the drug compounds, as well as data regarding the effectiveness of the drug compounds against certain diseases. The structural information for the drug compounds may comprise simplified molecular-input line-entry system (SMILES) strings. In an embodiment, the biological data may include datasets of genetic variation data, somatic mutation data, electronic health records, pathway enrichment data, gene expression data, protein expression data, disease ontology data, protein interactions data, and/or various scores/ranking associated with the drug compounds. The input data associated with each drug compound may be represented as an array or a vector. An input vector or array may comprise structured data, a dataset, a mathematical object, or a list of values that represent the drug data and biological data for a drug compound. In certain embodiments, the input vector may represent a combination of the drug molecular-structure data and the drug biological-treatment data for a drug compound.

The variational auto-encoder may compress the input vectors for each drug compound based on attributes or correlations determined from the data during training. In an embodiment, the output of a variational auto-encoder may describe the metrics, such as probability measurements from multivariate Gaussian distributions, for latent attributes of drug compounds. The metrics may be represented as a latent space, which may comprise embedding or encoding vectors that describe the probability metrics for a plurality of drug compounds and their attributes. In an embodiment, the elements of an embedding vector may represent the probability metrics for the latent attributes for a drug compound. A decoder may randomly sample from the metrics for desired attributes, and generate reconstruction vectors that may comprise structured data in a form similar to that of the input vectors that may be utilized to identify candidate drug compounds.

In certain embodiments, the aforementioned interpolation methods may be used to explore the latent space to determine and define the boundaries of an interpolation region to be further analyzed in order to identify candidate drug compounds. In some embodiments, the interpolation space or region of interest may be identified based on linear and spherical interpolation paths determined using the interpolation methods. The rankings of the candidate points within each interpolation region may be determined using a QSAR model. In embodiment, the ranking may be based on the embedding vectors of the drug compounds and a biomarker. In some embodiments, the rankings of the drug compounds may be based on a predicted target-value for the compounds, such as a binding activity, toxicity, and/or efficacy value. In certain embodiments, a vector path between the two clusters may be determined based on the ranking of the compounds within each interpolation region.

In an embodiment, the vector path may represent prime compounds that may be optimal candidates for experimental usage in treating certain diseases. Further, in accordance with some embodiments, the benefits of this disclosure may include the discovery of new molecular formulation for existing drugs, and a reduction in the time spent during experimental testing by identifying optimal outputs. Embodiments of the present disclosure may enable a system/platform where a user may input their drug data and receive drug variations to test.

### BRIEF DESCRIPTION OF THE DRAWINGS

The foregoing and other objects, features, and advantages for embodiments of the present disclosure will be apparent from the following more particular description of the embodiments as illustrated in the accompanying drawings, in which reference characters refer to the same parts throughout the various views. The drawings are not necessarily to scale, emphasis instead being placed upon illustrating principles of the present disclosure.

FIGS. 1(a)-(g) are exemplary graphs illustrating latent space exploration at certain steps of an embodiment for determining a vector path between two clusters of nodes representing metrics for drug compounds associated with the biological-treatment of diseases, in accordance with certain embodiments of the present disclosure.

FIG. 2 is a block diagram illustrating an embodiment of a system for implementing latent space exploration, in accordance with certain embodiments of the present disclosure.

FIG. 3 is a block diagram illustrating an embodiment of a computer architecture for a computer device for implementing latent space exploration, in accordance with certain embodiments of the present disclosure.

FIG. 4 is a flowchart illustrating the use of latent space exploration to identify a new molecule that may potentially be used as new drug, in accordance with certain embodiments of the present disclosure.

FIG. 5 is a flowchart illustrating steps of an embodiment for identifying top-ranked molecules that may potentially be used for experimental usage in the treatment of diseases, in accordance with certain embodiments of the present disclosure.

FIG. 6 is a flowchart illustrating steps of an embodiment for determining a prime drug compound for the treatment of a disease, in accordance with certain embodiments of the present disclosure.

### DETAILED DESCRIPTION OF EMBODIMENTS OF THE INVENTION

Reference will now be made in detail to the embodiments of the present disclosure, examples of which are illustrated in the accompanying drawings.

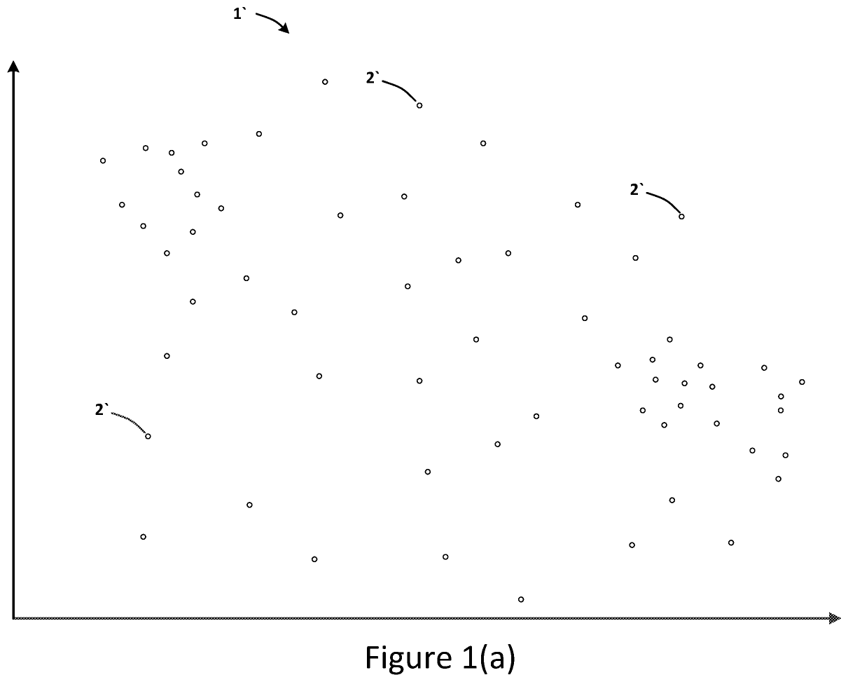

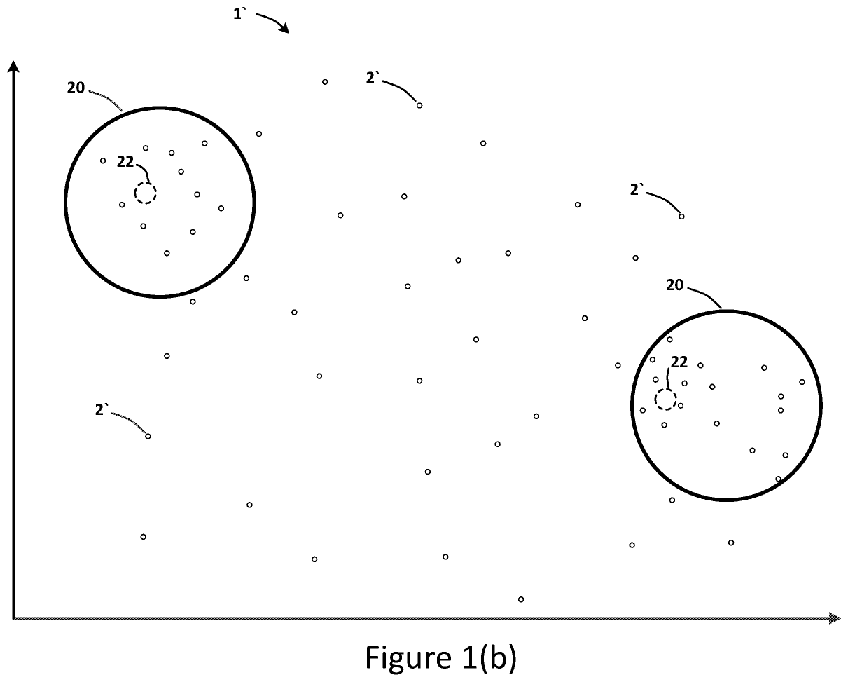

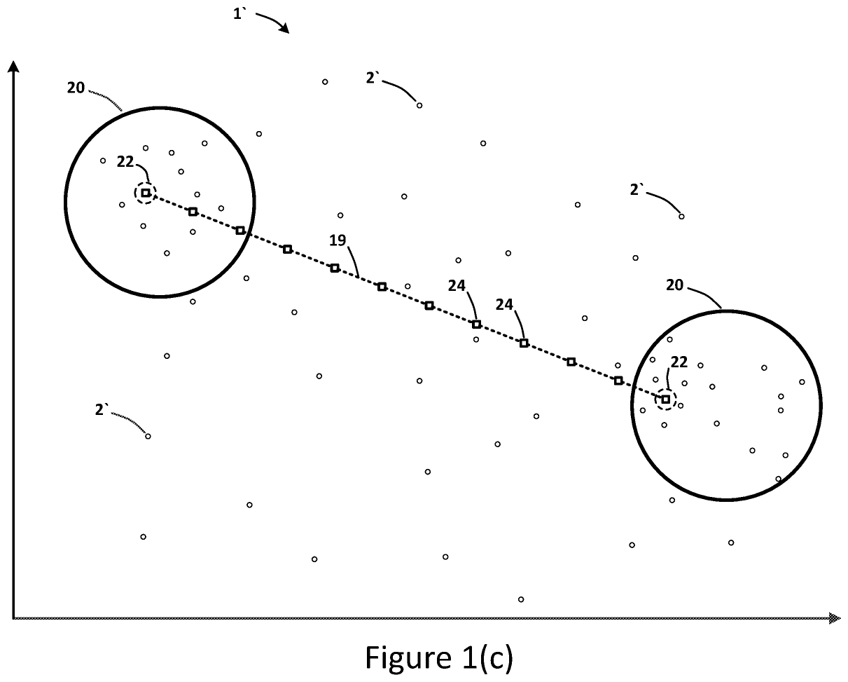

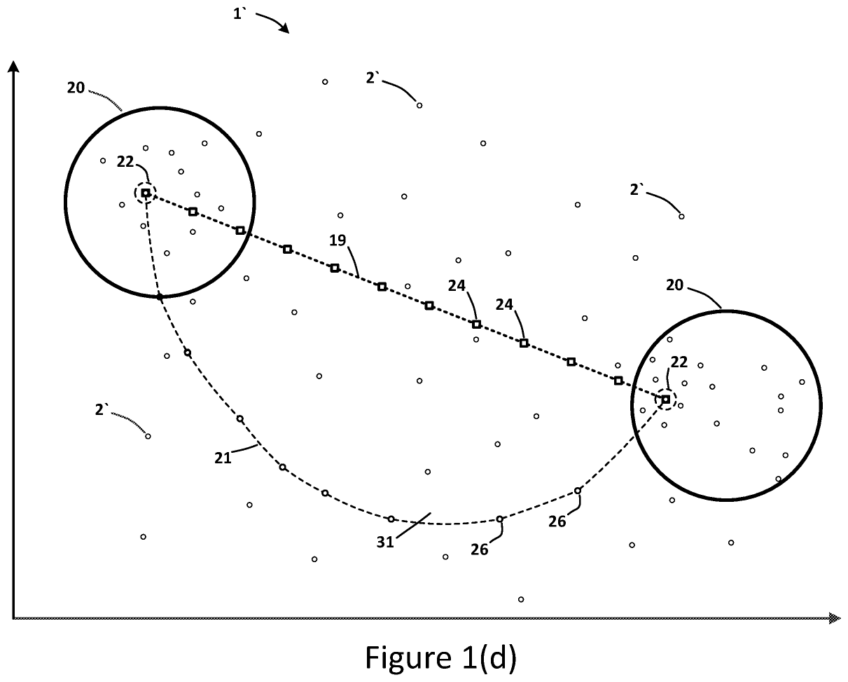

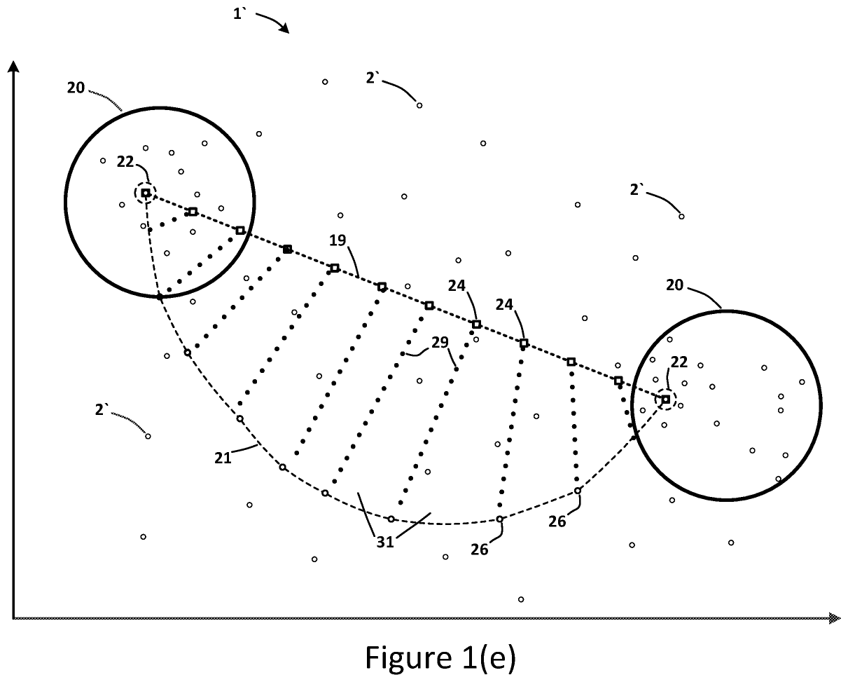

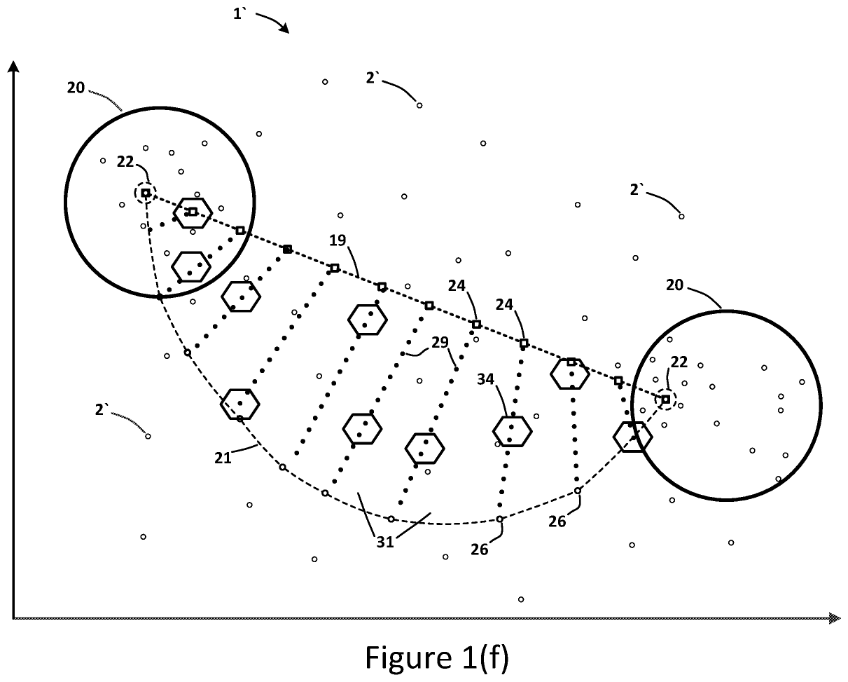

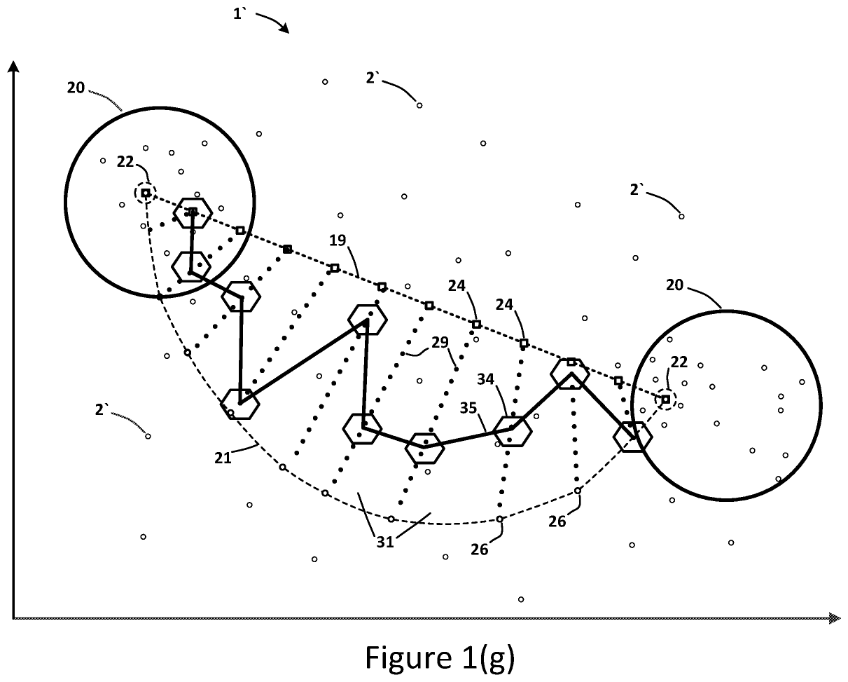

The present disclosure may be embodied in various forms, including a product, a system, a method or a computer readable medium for latent space exploration of a dataset based on drug molecular-structure data and drug biological-treatment data for a set of drug compounds in order to identify drug compounds having desired properties for treating diseases. A latent space 1 may be represented as a graphical plot of embedding vectors 2 representing metrics, such as probability metrics, for a set of drug compounds 3 and certain properties or attributes of the drug compounds 3. FIGS. 1(a)-(g) illustrate exemplary graphs representing the exploration of a latent space 1, in accordance with certain embodiments. FIG. 1(a) illustrates an exemplary graph 1′ corresponding to the latent space 1, wherein each node 2′ shown in the graph 1′ may correspond to an embedding vector 2 of the latent space 1.

Embedding vectors 2 may be generated using an encoder 4, such as a variational auto-encoder (VAE) 4, based on input data 5 representative of the drug compounds 3. In an embodiment, an input vector 5 may be based on a combination of molecular-structure data 6 and biological-treatment data 7 corresponding to that drug compound 3. For example, SMILES strings 6 and biological data 7 may be converted into vector representations that may be combined to generate embedding vectors 2. The variational auto-encoder 4 may be trained so that the embedding vectors 2 map, or correlate, to the input vectors 5.

Computations may be performed on the embedding vectors 2 in the latent space 1, such as regional interpolation 8 and the decoding of random vectors 2 that are likely to correspond to desired attributes 9 for drug compounds 3. In some embodiments, the latent space 1 comprises embedding vectors 2 describing or representing metrics 10 (e.g., probability metrics 10) for drug compounds 3, as well as their associated latent attributes 9, having certain molecular-structure data 6 and certain biological-treatment data 7 that relate to certain diseases 11 to be treated. In an embodiment, embedding vectors 2 corresponding to drug compounds 3 having a predetermined biomarker 12 of interest may be targeted. Embedding vectors 2 may be targeted based on certain values 13 for the attributes 9, such as a predetermined binding activity, toxicity, or efficacy value for a drug compound 3. The embedding vectors 2 may be ranked based on such target values 13, and decoded to identify the top-ranked drug compounds 3 for further experimental testing in laboratories 14.

In an embodiment, a method may include an initial step of receiving drug molecular-structure data 6 and drug biological-treatment data 7 from various databases. Such received data 15 may be combined into a dataset 5 (e.g., an input vector 5) that may be converted via an encoder 4 into an embedding dataset 2 (e.g., an embedding vector 2) represented in a latent space 1. In certain embodiments, the encoder 4 may comprise a variational auto-encoder 4. In accordance with some embodiments, the method may include the step of receiving a drug molecular-structure query 16, a drug treatment query 17, and a drug effect query 18. The method may include the step of determining a linear interpolation path 19 between clusters 20 of embedding vectors 2 in the latent space 1. FIG. 1(b) illustrates such clusters 20, and FIG. 1(c) depicts an exemplary linear interpolation path 19. The method may include the step of determining a curved or non-linear interpolation path 21 (such as a spherical, circular or elliptical path 21) between such clusters 20 in the latent space 1, as shown in FIG. 1(d).

In accordance with some embodiments, the determination of the linear interpolation path 19 and the determination of the non-linear interpolation path 21 may be based on one or more queries 16-18. For example, the queries may comprise the drug molecular-structure query 16, the drug treatment query 17, and the drug effect query 18. In an embodiment, the targeted clusters 20 of embedding vectors 2 may have metrics 10 for attributes 9 of drug compounds 3 that are greater than a predetermined value, such that the clusters 20 of embedding vectors 2 are determined to be responsive to the drug molecular-structure query 16, the drug treatment query 17, and/or the drug effect query 18. In order words, the targeted clusters 20 comprise a region of embedding vectors 2 having a high probability for a desired attribute 9 that corresponds to a query 16-18. In an embodiment, the interpolation paths 19 and 21 may extend from the centroid 22 of a first cluster 20 to the centroid 22 of a second cluster 20, as shown in FIG. 1(d). Accordingly, the targeted clusters 20 in the latent space 1 may represent drug compounds 3 that correspond to two diseases 11 to be treated. Such diseases 11 may be targeted or predetermined based on the drug molecular-structure query 16, the drug treatment query 17, and/or the drug effect query 18.

In certain embodiments, clusters in the latent space 1 may correspond to metrics 10 for attributes 9 of drug compounds 3 that may be associated with biological-treatment data 7 for specific diseases 11. In an embodiment, the method may include the step of annotating or marking the latent space 1 with disease labels 23. The disease labels 23 may correspond to diseases 11 that may be effectively treated by the drug compounds 3 represented by the corresponding clusters 20 in the latent space 1. The clusters 20 of embedding vectors 2 may be assigned to certain diseases 11, such as HIV or breast cancer.

In some embodiments, the method may include the steps of determining a first set of candidate points 24 on the linear interpolation path 19 based on a first predetermined stop-parameter 25. The method may include the step of determining a second set of candidate points 26 on the non-linear (e.g. spherical, circular or elliptical) interpolation path 21 based on the first predetermined stop-parameter 25. Accordingly, the two interpolation paths 19 and 21 may extend between the same two start and end points, e.g. the centroids 22 of the two clusters 20. FIG. 1(d) depicts the two sets of candidate points 24 and 26 on the two interpolation paths 19 and 21, extending between two centroids 22.

In certain embodiments, the method may further include the step of determining a linear chord interpolation path 28 between each candidate point 24 on the linear interpolation path 19 and each corresponding candidate point 25 on the non-linear interpolation path 21. The method may include the steps of determining a third set of candidate points 29 on each linear chord interpolation path 28 based on a second predetermined stop-parameter 30, and the step of determining an interpolation region 31 bound by the interpolation paths 19 and 21. In certain embodiments, the candidate points 24, 26 and 29 may comprise nodes 2′ located within the interpolation region 31. FIG. 1(e) illustrates the candidate points 29 for the linear chord interpolation paths 28 within the interpolation region 31. The method may determine a drug effect score 32 of each of the first set of candidate points 24, each of the second set of candidate points 26, and each of the third set of candidate points 29 using a quantitative structure-activity relationship (QSAR) model 33. In accordance with certain embodiments, as shown in FIG. 1(f), the method may further include the step of determining prime or optimal candidate points 34 within the interpolation region 31, e.g. the highest ranked candidate points 29 on each linear chord interpolation path 28 based on the drug effect scores 32.

In an embodiment, the optimal candidate points may be the top-ranked nodes 2′ within the interpolation region 31. This may include any of the candidate points 24, 26 and 29, as well as any other interpolation points within their boundaries, that are determined to have the highest rank at each iteration or step of the quantitative structure-activity relationship (QSAR) model 33. Using a linear chord interpolation paths 28 that links the linear interpolation path 19 and the non-linear interpolation path 21, step changes between a first cluster 20 and a second cluster 20 of nodes 2′ in the graphically represented latent space 1 may be determined. At each step between the two clusters 20, the highest ranked node 2′, which may be an interpolation point (e.g., a representation that may correspond to a new drug compound) or an embedding vector (e.g., a representation that may correspond to a preexisting drug compound), may be determined.

In an embodiment, the ranking determination may identify the top-ranked candidate points 29 on each linear chord interpolation path 28. FIG. 1(g) illustrates a vector path 35 within the interpolation region 31 based on the rankings of the third set of candidate points 29. In certain embodiments, the vector path 35 may comprise any node 2′ located within the interpolation region 31, including any interpolation point or embedding vector in the interpolation region 31. The visual representation of the vector path 35 from one step of the latent space exploration to the next between the two clusters 20 may resemble a “walk” up a staircase defined the linear interpolation path 19 and the non-linear interpolation path 21 on two sides and the linear chord interpolation paths 28 at each edge of the staircase steps. In certain embodiments, the ranking of nodes 2′ that graphically represent the latent space 1 may be based on a comparison of the drug effect scores 32. In an embodiment, the method may include the step of determining the optimal or prime drug molecular-structures 36 based on the prime candidate points 34. This determination may be performed with a neural network using an encoder 4 and decoder 40.

In some embodiments, the prime drug molecular-structures 36 may be determined using a variational auto-encoder (VAE) 4 and decoder 40. The variational auto-encoder 4 may generate embedding vectors 2 that represent probability measurements or metrics. The variational auto-encoder 4 may be denoted as qθ(z|x), and the decoder 40 may be denoted as pθ(x|z). In an embodiment, the input for the encoder 4 may be a dataset x and the output may be a hidden representation z, while the input for the decoder 40 may be the representation z (e.g., the latent space 1) and the output may be the dataset x (e.g., parameters to the probability distribution of the input data 5). The variational auto-encoder 4 and its corresponding decoder 40 may have weights and biases θ. The variational auto-encoder 4 may generate samples from a latent space 1 according to some underlying, learned distribution. This may include mean and standard deviation values. As such, the step of generating an embedding vector 2 that represents a metric 10 may be analogous to sampling from a distribution.

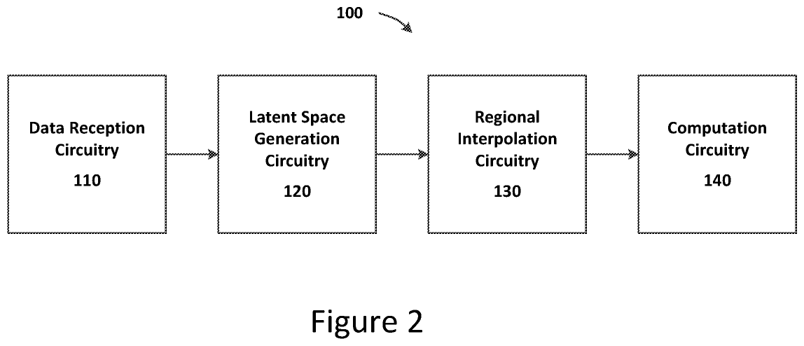

FIG. 2 illustrates an embodiment of a system 100 for implementing latent space exploration. The circuitry described herein may include the hardware, software, middleware, application program interfaces (APIs), and/or other components for implementing the corresponding features of the circuitry. Initially, a data reception circuitry 110 may be configured to receive drug molecular-structure data 6 and drug biological-treatment data 7. In an embodiment, a latent space generation circuitry 120 may be configured to combine the received molecular-structure data 6 and the received biological-treatment data 7 into a combined dataset 5, e.g. an input vector 5, represented in a latent space 1 using a variational auto-encoder 4. The latent space generation circuitry 120 may compress the input data 5 by applying convolutional neural-network layers and encoder models, e.g. variational auto-encoders (VAEs) 4 models, and generate embedding vectors 2 that represent metrics 10 for latent attributes 9. Accordingly, the latent space generation circuitry 120 may construct a latent space 1 from the received information, in accordance with certain embodiments. The resulting graphical representation 1′ may illustrate structured data having a specific format where each point or node 2′ may represent metrics 10 for latent attributes 9 that may correspond to a drug compound 3.

The latent space exploration system 100 may further include a regional interpolation circuitry 130 that may be configured to: determine a linear interpolation path 19 between clusters 20 of embedding vectors 2 in the latent space 1; determine a curved or non-linear (e.g., spherical, circular or elliptical) interpolation path 21 between clusters 20 of embedding vectors 2 in the latent space 1; determine a first set of candidate points 24 on the linear interpolation path 19 based on a first predetermined stop-parameter 25; determine a second set of candidate points 26 on the non-linear interpolation path 21 based on the first predetermined stop-parameter 25; determine a linear chord interpolation path 28 between each candidate point 24 on the linear interpolation path 19 and each corresponding candidate point 26 on the non-linear interpolation path 21; and, determine a third set of candidate points 29 on each linear chord interpolation path 28 based on a second predetermined stop-parameter 30. The regional interpolation circuitry 130 may determine an interpolation region 31 bound by the interpolation paths 19 and 21.

In some embodiments, the system may include a computation circuitry 140 configured to apply a QSAR model 33 to the embedding vectors 2 in an interpolation region 31 of the latent space 1. The computation circuitry 140 may determine a drug effect score 32 of each of the first plurality of candidate points 24, each of the second plurality of candidate points 26, and each of the third plurality of candidate points 29 using the quantitative structure-activity relationship model 33. The computation circuitry 140 may further determine prime candidate points 34 based on the drug effect scores 32, and determine prime drug molecular structures 36 based on the prime candidate points 34. Overall, executing the latent space exploration process provides improvements to the computing capabilities of a computer device executing the process by reducing the search space and by allowing for more efficient data analysis in order to analyze large amounts of data in a shorter amount of time.

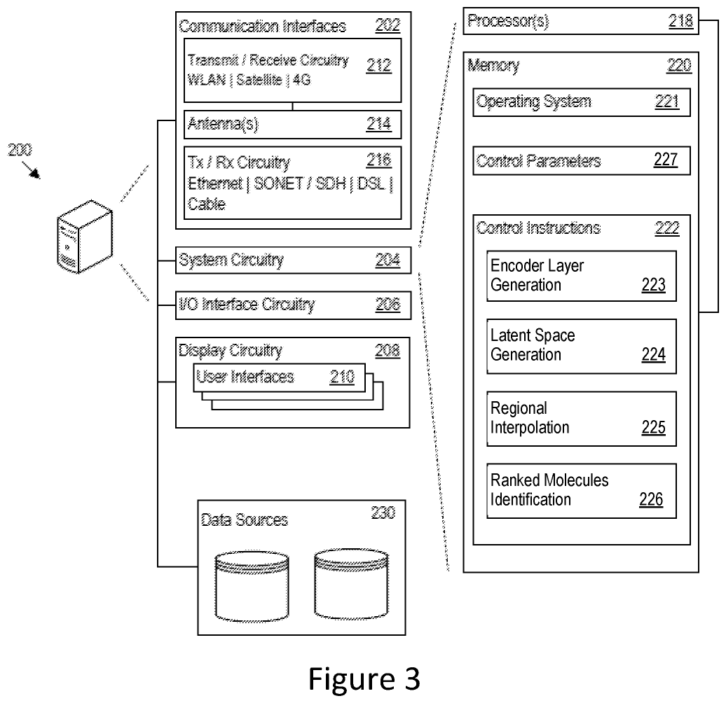

FIG. 3 illustrates an exemplary computer architecture of a computer device 200 on which the features of the latent space exploration system 100 may be executed. The computer device 200 includes communication interfaces 202, system circuitry 204, input/output (I/O) interface circuitry 206, and display circuitry 208. The graphical user interfaces (GUIs) 210 displayed by the display circuitry 208 may be representative of GUIs generated by the system 100 to present a query to an enterprise application or end user, requesting information on a compound 3 to be replaced and/or compound attributes desired to be satisfied by a candidate discovery compound. The graphical user interfaces (GUIs) 210 displayed by the display circuitry 208 may also be representative of GUIs generated by the system 100 to receive query inputs 16-18 identifying the compound 3 to be replace and/or compound attributes 9 desired to be satisfied by a candidate discovery compound. The GUIs 210 may be displayed locally using the display circuitry 208, or for remote visualization, e.g., as HTML, JavaScript, audio, and video output for a web browser running on a local or remote machine. Among other interface features, the GUIs 210 may further render displays of any new formulations resulting from the replacement of compounds(s) 3 with discovery compound(s) selected from the processes described herein.

The GUIs 210 and the I/O interface circuitry 206 may include touch sensitive displays, voice or facial recognition inputs, buttons, switches, speakers and other user interface elements. Additional examples of the I/O interface circuitry 206 includes microphones, video and still image cameras, headset and microphone input/output jacks, Universal Serial Bus (USB) connectors, memory card slots, and other types of inputs. The I/O interface circuitry 206 may further include magnetic or optical media interfaces (e.g., a CDROM or DVD drive), serial and parallel bus interfaces, and keyboard and mouse interfaces.

The communication interfaces 202 may include wireless transmitters and receivers (“transceivers”) 212 and any antennas 214 used by the transmit-and-receive circuitry of the transceivers 212. The transceivers 212 and antennas 214 may support WiFi network communications, for instance, under any version of IEEE 802.11, e.g., 802.11n or 802.11ac, or other wireless protocols such as Bluetooth, Wi-Fi, WLAN, cellular (4G, LTE/A). The communication interfaces 202 may also include serial interfaces, such as universal serial bus (USB), serial ATA, IEEE 1394, lighting port, I²C, slimBus, or other serial interfaces. The communication interfaces 202 may also include wireline transceivers 216 to support wired communication protocols. The wireline transceivers 216 may provide physical layer interfaces for any of a wide range of communication protocols, such as any type of Ethernet, Gigabit Ethernet, optical networking protocols, data over cable service interface specification (DOCSIS), digital subscriber line (DSL), Synchronous Optical Network (SONET), or other protocol.

The system circuitry 204 may include any combination of hardware, software, firmware, APIs, and/or other circuitry. The system circuitry 204 may be implemented, for example, with one or more systems on a chip (SoC), application specific integrated circuits (ASIC), microprocessors, discrete analog and digital circuits, and other circuitry. The system circuitry 204 may implement any desired functionality of the system 100. As just one example, the system circuitry 204 may include one or more instruction processor 218 and memory 220.

The memory 220 stores, for example, control instructions 222 for executing the features of the system 100, as well as an operating system 221. In one implementation, the processor 218 executes the control instructions 222 and the operating system 221 to carry out any desired functionality for the system 100, including those attributed to encoder layer generation 223 and latent space generation 224 (e.g., relating to the latent space generation circuitry 120), regional interpolation 225 (e.g., relating to the regional interpolation circuitry 130), and/or ranked molecules identification 226 (e.g., relating to the computation circuitry 140). The control parameters 227 provide and specify configuration and operating options for the control instructions 222, operating system 221, and other functionality of the computer device 200.

The computer device 200 may further include various data sources 230. Each of the databases that are included in the data sources 230 may be accessed by the system 100 to obtain data for consideration during any one or more of the processes described herein. For example, the data reception circuitry 110 may access the data sources 230 to receive the input data for generating the latent space 1.

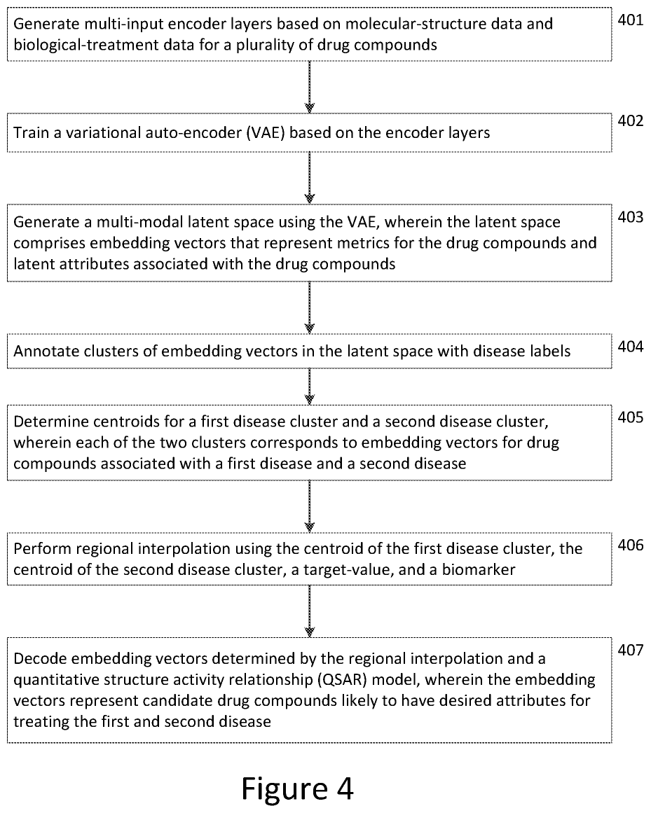

FIGS. 4-6 show flowcharts representing exemplary processes or methods implemented by the system 100, in accordance with certain embodiments. The processes may be implemented by a computing device 200, system, and/or circuitry components as described herein.

In an embodiment, as set forth in block 401 of FIG. 4, a system 100 may generate multi-input encoder layers 37 based on molecular-structure data 6 and biological-treatment data 7 for a plurality of drug compounds 3. As noted above, this step for encoder layer generation 223 may be implemented by the latent space generation circuitry 120. The system 100 may further train a variational auto-encoder (VAE) 4 based on the encoder layers 37 (block 402), and generate a multi-modal latent space 1 using the variational auto-encoder 4 (block 403). The latent space 1 may comprise embedding vectors 2 that represent metrics 10 for the drug compounds 3 and the latent attributes 9 associated with the drug compounds 3. In some embodiments, as depicted by block 404, the system 100 may annotate clusters 20 of embedding vectors 2 in the latent space 1 with disease labels 23. The system 100 may also determine centroids 22 for a first disease cluster 20 and a second disease cluster 20, wherein each of the two clusters 20 corresponds to embedding vectors 2 for drug compounds 3 associated with a first disease 11 and a second disease 11, as set forth in block 405. In an embodiment, the first disease 11 may be associated with HIV and the second disease 11 may be associated with breast cancer.

In addition, the system 100 may perform regional interpolation (block 406) using the centroid 22 of the first disease cluster 20, the centroid 22 of the second disease cluster 20, a target-value 13, and a biomarker 12. This step for regional interpolation 225 may be implemented by the regional interpolation circuitry 130. In an embodiment, the target-value 13 may comprise a binding activity value greater than the value of 5. In some embodiments, the biomarker 12 may represent a high gene expression in genes related to breast cancer or inflammation. As depicted in block 407, via the computation circuitry 140, a system 100 may decode embedding vectors 2 determined by the regional interpolation and a quantitative structure activity relationship (QSAR) model 33, wherein the embedding vectors 2 represent candidate drug compounds 3 likely to have desired attributes 9 for treating the first and second disease 11.

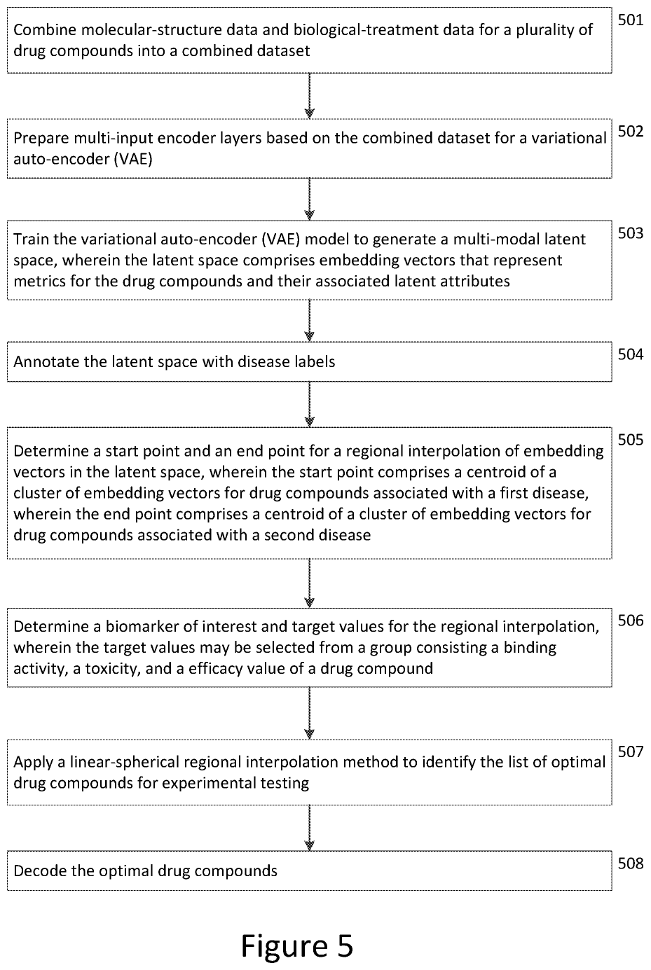

FIG. 5 illustrates an exemplary process implemented by a system 100 that may combine (block 501) molecular-structure data 6 and biological-treatment data 7 for a plurality of drug compounds 3 into a combined dataset 5, in accordance with certain embodiments. The system 100 may further prepare multi-input encoder layers 37 based on the combined dataset 5 for a variational auto-encoder 4 (block 502). As depicted in block 503, the system 100 may train the variational auto-encoder model 4 to generate a multi-modal latent space 1, wherein the latent space 1 comprises embedding vectors 2 that represent metrics 10 for latent attributes 9 associated with the drug compounds 3 and their latent attributes 9. The system 100 may also annotate (block 504) the latent space 1 with disease labels 23.

Further, the system 100 may determine a start point 38 and an end point 39 for a regional interpolation of embedding vectors 2 in the latent space 1 (block 505). The start point 38 may comprise a centroid 22 of a cluster 20 of embedding vectors 2 for drug compounds 3 associated with a first disease 11, and the end point 39 may comprise a centroid 22 of a cluster 20 of embedding vectors 2 for drug compounds 3 associated with a second disease 11. In an embodiment, the first disease 11 may be associated with HIV and the second disease 11 may be associated with cancer.

The system 100 may determine a biomarker 12 of interest and target values 13 for the regional interpolation (block 506). The target values 13 may include a binding activity value, a toxicity value, and a efficacy value of a drug compound 3. Block 507 depicts the application of a linear-spherical regional interpolation method by the system 100 in order to identify the list of optimal drug compounds 3 for experimental testing. The system 100 may further decode (block 508) the optimal drug compounds 3.

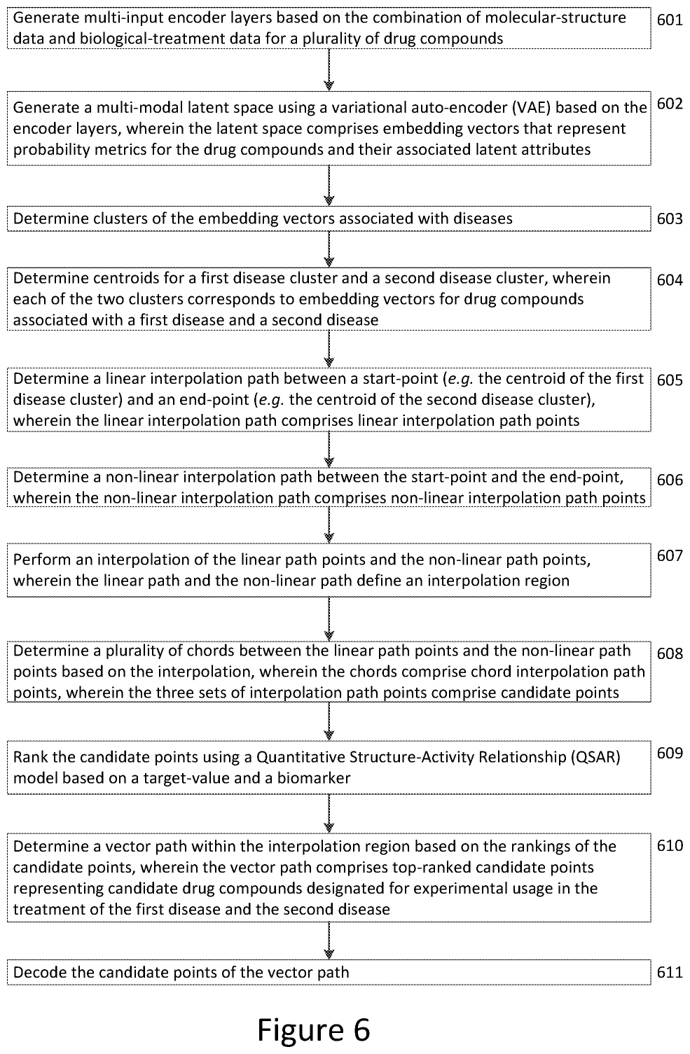

As illustrated in FIG. 6, an embodiment of a system 100 may implement an exemplary process or method that generates (block 601) multi-input encoder layers 37 based on the combination of molecular-structure data 6 and biological-treatment data 7 for a plurality of drug compounds 3. At block 602, the system 100 may generate a multi-modal latent space 1 using a variational auto-encoder 4 based on the encoder layers 37. The latent space 1 may comprise embedding vectors 2 that represent metrics 10 for the drug compounds 3 and latent attributes 9 associated with the drug compounds 3. The system 100 may further determine clusters 20 of the embedding vectors 2 associated with diseases 11 (block 603), and determine centroids 22 for a first disease cluster 20 and a second disease cluster 20. Each of the two clusters 20 may correspond to embedding vectors 2 for drug compounds 3 associated with a first disease 11 and a second disease 11 (block 604).

As depicted in block 605, the system 100 may determine a linear interpolation path 19 between a start-point 38 (e.g., the centroid 22 of the first disease cluster 20) and an end-point 39 (e.g., the centroid 22 of the second disease cluster 20). The linear interpolation path 19 may comprise linear interpolation path points 24. In an embodiment, the linear interpolation path points 24 comprise the aforementioned first set of candidate points 24 on the linear interpolation path 19 that are based on the first predetermined stop-parameter 25. Further, the system 100 may determine a non-linear interpolation path 21 between the start-point 38 and the end-point 39, wherein the non-linear interpolation path 21 comprises non-linear interpolation path points 26 (block 606). In an embodiment, the non-linear interpolation path points 26 comprise the aforementioned second set of candidate points 26 on the linear interpolation path 21 that are based on the first predetermined stop-parameter 25.

As set forth in block 607, the system 100 may perform an interpolation of the linear interpolation path points 24 and the non-linear interpolation path points 26, wherein the linear interpolation path 19 and the non-linear interpolation path 21 define an interpolation region 31. In addition, the system 100 may determine a plurality of chords 28 between the linear interpolation path points 24 and the corresponding non-linear interpolation path points 26 based on the interpolation (block 608), wherein the chords 28 comprise a third set of candidate points 29. The system 100 may rank (block 609) the candidate points 24, 26 and 29 using a Quantitative Structure-Activity Relationship (QSAR) model 33 based on a target-value 13 and a biomarker 12. To provide additional context of the technical field and the QSAR model 33 disclosed herein, the contents of U.S. Pat. No. 10,301/273, which issued on May 28, 2019, that describe QSAR methods are hereby incorporated by reference herein. Further, the system 100 may determine a vector path 35 within the interpolation region 31 based on the rankings of the candidate points 24, 26 and 29 (block 610). In an embodiment, the vector path 35 may comprise top-ranked candidate points 24, 26 and 29 representing candidate drug compounds 3 designated for experimental usage in the treatment of the first disease 11 and the second disease 11. The system 100 may also decode (block 611) the candidate points 24, 26 and 29 of the vector path 35.

The interpolation implemented by embodiments of the disclosed systems and methods may include a linear interpolation (LERP) operation and a spherical linear interpolation (SLERP) operation. A number of intermediate points 24 along the linear interpolation path 19 may generated. Setting a parameter t equal to 10, the LERP interpolation may generate ten intermediate points 24 along the linear interpolation path 19 using the following function with multivariate input data 5 denoted as v₀, and v₁:

LERP(v₀, v₁, t)=v₀+t(v₁−v₀)

A number of intermediate points 26 may generated along a spherical interpolation path 21. Setting a parameter t equal to 10, the SLERP interpolation may generate ten intermediate points 26 along the linear interpolation path 21 using the following function with multivariate input data 5 denoted as v₀, and v₁:

$$SLERP ( {v}_{0} , {v}_{1} , t ) = \frac{sin ( ( 1 - t ) θ )}{sin θ} {v}_{0} + \frac{sin ( t θ )}{sin θ} {v}_{1}$$

The SLERP path 21 is the spherical geometry equivalent of a path along the LERP path 19. When the end vectors are perpendicular, the operation may comprise the parametric circle formula, in accordance with certain embodiments:

\vec{c}=(cos θ)\hat{x}+(sin θ)ŷ=(cos θ)v₀+(sin θ)v₁

In another embodiment, the non-linear interpolation path 21 may elliptical. Such a non-linear interpolation path 21 may be generated using the following function, wherein each component may be scaled to the lengths of the semi-major and semi-minor axes of the ellipse, α and β respectively:

=α(cos θ)\hat{x}+(sin θ)ŷ=(cos θ)v₀+β(sin θ)v₁

While the present disclosure has been particularly shown and described with reference to an embodiment thereof, it will be understood by those skilled in the art that various changes in form and details may be made therein without departing from the spirit and scope of the present disclosure. Although some of the drawings illustrate a number of operations in a particular order, operations that are not order-dependent may be reordered and other operations may be combined or broken out. While some reordering or other groupings are specifically mentioned, others will be apparent to those of ordinary skill in the art and so do not present an exhaustive list of alternatives.

What is claimed is:

1. A method for latent space exploration based on drug molecular-structure data and drug biological-treatment data to determine a drug compound for treating diseases, comprising the steps of:4
generating multi-input encoder layers based on molecular-structure data for a plurality of drug compounds and on biological-treatment data for the plurality of drug compounds;
generating a multi-modal latent space using a variational auto-encoder based on the encoder layers, wherein the latent space comprises embedding vectors, and wherein each embedding vector is a metric representation of the plurality of drug compounds and a plurality of attributes associated with the drug compounds;
determining clusters of the embedding vectors in the latent space associated with diseases;
determining centroids for a first disease cluster and a second disease cluster, wherein each of the two clusters corresponds to embedding vectors for drug compounds associated with a first disease and a second disease;
determining a linear interpolation path between a start-point and an end-point, wherein the start-point comprises the centroid of the first disease cluster, wherein the end-point comprises the centroid of the second disease cluster, wherein the linear interpolation path comprises linear interpolation path points;
determining a non-linear interpolation path between the start-point and the end-point, wherein the non-linear interpolation path comprises non-linear interpolation path points, wherein the linear interpolation path and the non-linear interpolation path define an interpolation region;
performing an interpolation of the linear interpolation path points and the non-linear interpolation path points;
determining a plurality of chords between the linear interpolation path points and the non-linear interpolation path points based on the interpolation, wherein the chords comprise chord interpolation path points, and wherein the linear interpolation path points, the non-linear interpolation path points and the chord interpolation path points comprise candidate points;
ranking the candidate points using a quantitative structure-activity relationship model based on a target-value and a biomarker;
determining a vector path within the interpolation region based on the rankings of the candidate points, wherein the vector path comprises top-ranked candidate points representing candidate drug compounds designated for experimental usage in the treatment of the first disease and the second disease; and, decoding the top-ranked candidate points of the vector path.

2. The method of claim 1, wherein the molecular-structure data comprises simplified molecular-input line-entry system (SMILES) strings for the plurality of drug compounds.

3. The method of claim 1, wherein the biological-treatment data is selected from a group consisting of: genetic variation data, somatic mutation data, electronic health records data, pathway enrichment data, gene expression data, protein expression data, disease ontology data, and protein interactions data.

4. The method of claim 1, wherein the biological-treatment data comprises genetic variation data, somatic mutation data, electronic health records data, pathway enrichment data, gene expression data, protein expression data, disease ontology data, and protein interactions data.

5. The method of claim 1, wherein the target-value is selected from a group consisting of: binding activity, toxicity, and efficacy.

6. The method of claim 1, wherein the non-linear interpolation path is selected from a group consisting of: a spherical path, a circular path, and an elliptical path.

7. The method of claim 1, wherein the step of determining clusters associated with diseases comprises the steps of:
performing a query of the embedding vectors in the latent space to identify the clusters, wherein the query is selected from a group consisting of a drug molecular-structure query, a drug treatment query, and a drug effect query; and,
annotating the embedding vectors of the clusters with disease labels, wherein the disease labels correspond to the first disease and the second disease.

8. A method, comprising the steps of:
receiving drug molecular-structure data corresponding to a plurality of drug compounds;
receiving drug biological-treatment data corresponding to the plurality of drug compounds;
combining the received molecular-structure data and the received biological-treatment data into a combined dataset;
receiving a drug molecular-structure query, a drug treatment query, and a drug effect query;
generating multi-input encoder layers based on the combined dataset;
generating a multi-modal latent space using a variational auto-encoder based on the encoder layers, wherein the latent space comprises embedding vectors, and wherein each embedding vector is a metric representation of the plurality of drug compounds and a plurality of attributes associated with the drug compounds;
determining a linear interpolation path between clusters of the embedding vectors in the latent space based on the drug molecular-structure query, the drug treatment query, and the drug effect query; and,
determining a non-linear interpolation path between clusters of the embedding vectors in the latent space based on the drug molecular-structure query, the drug treatment query, and the drug effect query.

9. The method of claim 8, wherein the non-linear interpolation path is selected from a group consisting of: a spherical path, a circular path, and an elliptical path.

10. The method of claim 9, furthering comprising the steps of:
determining a first plurality of candidate points on the linear interpolation path based on a first predetermined stop-parameter; and,
determining a second plurality of candidate points on the non-linear interpolation path based on the first predetermined stop-parameter.

11. The method of claim 10, furthering comprising the step of:
determining a linear chord path between candidate points on the linear interpolation path and corresponding candidate points on the non-linear interpolation path.

12. The method of claim 11, furthering comprising the steps of:
determining a third plurality of candidate points on each linear chord path based on a second predetermined stop-parameter; and,
determining a drug effect score of each of the first plurality of candidate points, each of the second plurality of candidate points, and each of the third plurality of candidate points using a quantitative structure-activity relationship model.

13. The method of claim 12, furthering comprising the step of:
determining prime candidate points based on the drug effect scores.

14. The method of claim 13, furthering comprising the step of:
determining a prime drug molecular structure based on the prime candidate points using the variational auto-encoder and a decoder.

15. A product for latent space exploration based on drug molecular-structure data and drug biological-treatment data to determine a drug compound for treating diseases, comprising:
a machine-readable medium, other than a transitory signal; and,
instructions stored on the machine-readable medium, the instructions configured to, when executed, cause processing circuitry to:
receive drug molecular-structure data corresponding to a plurality of drug compounds;
receive drug biological-treatment data corresponding to the plurality of drug compounds;
combine the received molecular-structure data and the received biological-treatment data into a combined dataset;
generate multi-input encoder layers based on the combined dataset;
generate a multi-modal latent space using a variational auto-encoder based on the encoder layers, wherein the latent space comprises embedding vectors, and wherein each embedding vector is a metric representation of the plurality of drug compounds and a plurality of attributes associated with the drug compounds;
determine a linear interpolation path between two clusters of the embedding vectors in the latent space; and,
determine a non-linear interpolation path between the two clusters of the embedding vectors in the latent space.

16. The product of claim 15, wherein the instructions, when executed, further cause the processing circuitry to:
assign disease labels to the two clusters of the embedding vectors in the latent space, wherein the two clusters comprise a first disease cluster and a second disease cluster;
determine centroids for the first disease cluster and the second disease cluster, wherein each of the two clusters corresponds to embedding vectors for drug compounds associated with a first disease and a second disease, wherein the centroid of the first disease cluster comprises a start-point for the linear interpolation path and the non-linear interpolation path, wherein the centroid of the second disease cluster comprises an end-point for the linear interpolation path and the non-linear interpolation path, wherein the linear interpolation path comprises linear interpolation path points, wherein the non-linear interpolation path comprises non-linear interpolation path points; wherein the linear interpolation path and the non-linear interpolation path define an interpolation region, and wherein the linear interpolation path points and the non-linear interpolation path points located within the interpolation region comprise candidate points;
perform an interpolation of the linear interpolation path points and the non-linear interpolation path points;
determine a plurality of chords between the linear interpolation path points and the non-linear interpolation path points based on the interpolation, wherein the chords comprise chord interpolation path points, and wherein the linear interpolation path points, the non-linear interpolation path points and the chord interpolation path points comprise candidate points;
rank the candidate points using a quantitative structure-activity relationship model based on a target-value and a biomarker;
determine a vector path within the interpolation region based on the rankings of the candidate points, wherein the vector path comprises top-ranked candidate points representing candidate drug compounds designated for experimental usage in the treatment of the first disease and the second disease; and,
decode the top-ranked candidate points of the vector path.

17. The product of claim 15, wherein the determination of the linear interpolation path and the determination of the non-linear interpolation path are based on a query, and wherein the two clusters correspond to embedding vectors for drug compounds having attributes identified by the query.

18. The product of claim 17, wherein the query is selected from a group consisting of: a drug molecular-structure query, a drug treatment query, and a drug effect query.

19. The product of claim 17, wherein the instructions, when executed, further cause the processing circuitry to:
determine a first plurality of candidate points on the linear interpolation path based on a first predetermined stop-parameter;
determine a second plurality of candidate points on the non-linear interpolation path based on the first predetermined stop-parameter;
determine a linear chord path between candidate points on the linear interpolation path and corresponding candidate points on the non-linear interpolation path;
determine a third plurality of candidate points on each linear chord path based on a second predetermined stop-parameter;
determine a drug effect score of each of the first plurality of candidate points, each of the second plurality of candidate points, and each of the third plurality of candidate points using a quantitative structure-activity relationship model;
determine prime candidate points based on the drug effect scores, wherein the prime candidate points represent candidate drug compounds designated for experimental usage in the treatment of the first disease and the second disease; and,
determine prime drug molecular structures based on the prime candidate points by decoding the prime candidate points.

20. A system for latent space exploration based on drug molecular-structure data and drug biological-treatment data to determine a drug compound for treating diseases, comprising:
a data reception circuitry, configured to:
receive drug molecular-structure data corresponding to a plurality of drug compounds;
receive drug biological-treatment data corresponding to the plurality of drug compounds;
a latent space generation circuitry, configured to:
combine the received molecular-structure data and the received biological-treatment data into a combined dataset;
generate multi-input encoder layers based on the combined dataset;
generate a multi-modal latent space using a variational auto-encoder based on the encoder layers, wherein the latent space comprises embedding vectors, and wherein each embedding vector is a metric representation of the plurality of drug compounds and a plurality of attributes associated with the drug compounds;
a regional interpolation circuitry, configured to:
determine a linear interpolation path between clusters of the embedding vectors in the latent space;
determine a non-linear interpolation path between clusters of the embedding vectors in the latent space;
perform an interpolation of linear interpolation path points of the linear interpolation path and non-linear interpolation path points of the non-linear interpolation path, wherein the linear interpolation path and the non-linear interpolation path define an interpolation region, wherein the linear interpolation path points and the non-linear interpolation path points located within the interpolation region comprise candidate points;
determine chords between points on the linear interpolation path and corresponding points on the non-linear interpolation path based on the interpolation, wherein the chords comprise chord interpolation path points, and wherein the linear interpolation path points, the non-linear interpolation path points and the chord interpolation path points comprise candidate points; and,
a computation circuitry, configured to:
rank the candidate points via a quantitative structure-activity relationship model using a target-value and a biomarker, wherein each of the ranked candidate points have a score determined by the quantitative structure-activity relationship model, wherein the candidate points are ranked based on the scores;
determine a vector path within the interpolation region based on the rankings of the candidate points, wherein the vector path comprises the candidate points having the largest score;
decode the candidate points of the vector path, wherein a prime drug molecular structure is determined for each candidate points of the vector path.
